(19) United States
(12) Patent Application Publication
(10) Pub. No.: US 2016/0123997 A1
(43) Pub. Date: May 5, 2016

(54) MATERIALS AND METHODS RELATING TO ALZHEIMER'S DISEASE

(71) Applicant: Electrophoretics Limited, Surrey (GB)

(72) Inventors: Hans Dieter Zucht, Hannover (DE); Ian Hugo Pike, Surrey (GB); Malcolm Andrew Ward, Surrey (GB)

(73) Assignee: Electrophoretics Limited, Surrey (GB)

(21) Appl. No.: 14/896,388

(22) PCT Filed: Jun. 5, 2014
(86) PCT No.: PCT/GB2014/051741

§ 371 Date: Dec. 6, 2015

(30) Foreign Application Priority Data
Jun. 7, 2013 (GB) 1310203.3

(51) Int. Cl. G01N 33/68

(52) U.S. Cl. CPC G01N 33/6896; G01N 2560/00; G01N 2800/60; G01N 2800/2821; G06F 19/24

(57) ABSTRACT

The invention relates to methods and compositions relating Alzheimer's disease. There is provided a panel of optimal biomarkers which allow diagnosis of Alzheimer's disease and discrimination between Alzheimer's disease and its earlier precursor, mild cognitive impairment (MCI).

### FIELD OF THE INVENTION

The present invention relates to methods and compositions relating to Alzheimer's disease. Specifically, the present invention identifies and describes optimal biomarker panels for the diagnosis of Alzheimer's disease and in particular allows discrimination between Alzheimer's disease and its earlier precursor, mild cognitive impairment (MCI).

### BACKGROUND OF THE INVENTION

Dementia is one of the major public health problems of the elderly, and in our ageing populations the increasing numbers of patients with dementia is imposing a major financial burden on health systems around the world. More than half of the patients with dementia have Alzheimer's disease (AD). The prevalence and incidence of AD have been shown to increase exponentially. The prevalence for AD in Europe is 0.3% for ages 60-69 years, 3.2% for ages 70-79 years, and 10.8% for ages 80-89 years (Rocca, Hofman et al. 1991). The survival time after the onset of AD is approximately from 5 to 12 years (Friedland 1993).

Alzheimer's disease (AD), the most common cause of dementia in older individuals, is a debilitating neurodegenerative disease for which there is currently no cure. It destroys neurons in parts of the brain, chiefly the hippocampus, which is a region involved in coding memories. Alzheimer's disease gives rise to an irreversible progressive loss of cognitive functions and of functional autonomy. The earliest signs of AD may be mistaken for simple forgetfulness, but in those who are eventually diagnosed with the disease, these initial signs inexorably progress to more severe symptoms of mental deterioration. While the time it takes for AD to develop will vary from person to person, advanced signs include severe memory impairment, confusion, language disturbances, personality and behaviour changes, and impaired judgement. Persons with AD may become non-communicative and hostile. As the disease ends its course in profound dementia, patients are unable to care for themselves and often require institutionalisation or professional care in the home setting. While some patients may live for years after being diagnosed with AD, the average life expectancy after diagnosis is eight years.

In the past, AD could only be definitively diagnosed by brain biopsy or upon autopsy after a patient died. These methods, which demonstrate the presence of the characteristic plaque and tangle lesions in the brain, are still considered the gold standard for the pathological diagnoses of AD. However, in the clinical setting brain biopsy is rarely performed and diagnosis depends on a battery of neurological, psychometric and biochemical tests, including the measurement of biochemical markers such as the ApoE and tau proteins or the beta-amyloid peptide in cerebrospinal fluid and blood.

Biomarkers, particularly those found in body fluids such as blood, plasma and cerebrospinal fluid may possibly possess the key in the next step for diagnosing AD and other dementias. A biological marker that fulfils the requirements for the diagnostic test for AD would have several advantages. An ideal biological marker would be one that is present in a readily accessible tissue such as plasma and that identifies AD cases at a very early stage of the disease, before there is degeneration observed in the brain imaging and neuropathological tests. A biomarker could be the first indicator for starting treatment as early as possible, and also very valuable in screening the effectiveness of new therapies, particularly those that are focussed on preventing the development of neuropathological changes. A biological marker would also be useful in the follow-up of the development of the disease. Biomarkers for use in the diagnosis of Azlheimer's disease have been identified previous (see for example U.S. Pat. No. 7,897,361 the contents of which are incorporated herein by reference). However, there exists a continuous need to provide more potent biomarkers which not only provide reliable results, but are able to distinguish between the different forms and stages of dementia, e.g. MCI and Alzheimer's disease. In this context, whilst reference is made to a biomarker this also includes the use of more than one biological marker within a pre-determined panel.

### SUMMARY OF THE INVENTION

The inventors have performed a novel quantitative mass spectrometric analysis of blood proteins extracted from blood plasma of age and sex matched patients with clinically diagnosed Alzheimer's disease, mild cognitive impairment and non-demented controls. Based on the relative abundance of 1,630 tryptic peptides between the three groups the inventors have created statistical models in order to select and prioritise plasma biomarkers for dementia. In doing so, they provide herein a panel of peptides having enhanced qualities as biomarkers for dementia such as Alzheimer's disease and its precursor MCI.

The inventors have created panels comprising multiple biomarkers which in combination improve the predications of disease, its progression and prognosis.

Accordingly, at its most general, the present invention provides methods of diagnosing Alzheimer's disease or MCI using biomarker panels comprising multiple peptides which have been selected based on statistical models such as polynomial regression model for increased prediction of type and stage of dementia in subjects.

Specifically, the inventors have determined combinations of peptide biomarkers that increase the prediction of Alzheimer's disease or MCI as compared to controls and as a result the inventors are able to provide improved methods in the diagnosis of forms and stages of dementia such as Alzheimer's disease and MCI.

In a first aspect the present invention provides a method of diagnosing Alzheimer's disease in a subject, the method comprising detecting the presence of two or more differentially expressed proteins using a biomarker panel comprising a combination of two or more peptides selected from Table 2, 3 or 4 in a tissue sample or body fluid sample from said subject. Preferably, the method is an in vitro method.

The combination of markers selected based on the mathematical modelling carried out by the inventors creates a biomarker panel with increased sensitivity and specificity over combinations of biomarkers provided in the art.

Indeed, the inventors have determined a set of 31 significant peptides (see Table 2) from a number of proteins (see Table 1) which may be used to not only diagnose Alzheimer's disease, but distinguish between this form of dementia and MCI and control subjects. Of these 31 peptides, the most relevant 30 were compiled into a 4 parametric AD model; a 2 parametric AD model; a 4 parametric MCI model and a 6 parametric MCI model (AD=Alzheimer's disease). Out of these, polynomial models were formed and the preferred combinations of biomarker peptides determined.

Tables 3 and 4 represent the most relevant variables which can be used to predict the occurrence of Alzheimer's disease or the presence of MCI. These Tables serve as a basis for a set of alternative panels where an arbitrary subset of two or more, preferably three or more, preferably four or more peptides can be selected. It is preferred that the subsets comprises at least two peptides having a higher attribute score (i.e. >15 usage or count). These peptides can then be complemented with further peptides having a lower score. Preferably all peptides selected for the subset will have a >15 attribute score (i.e. usage or count).

The inventors have further created a multimarker panel using group modelling and data handling (GMDH) algorithm. This technique produced a set of alternative panels or models, which are suitable for the diagnosis of Alzheimer's disease and MCI. The best 30 GMDH polynomial models for determining AD versus MCI and controls is provided in FIG. 5. The best 30 GMDH polynomial models for determining MCI versus AD and controls is provided in FIG. 7.

Accordingly, the present invention provides a method of diagnosing, assessing, and/or prognosing, Alzheimer's disease (AD) or MCI in a subject, the method comprising:
determining the presence or an amount (e.g. concentration) of a panel of biomarkers, said panel comprising two or more peptides selected from Table 2, Table 3 or Table 4 in a biological sample obtained from said subject, wherein
(a) the presence of said two or more peptides in said sample is indicative of the subject having Alzheimer's disease;
(b) the amount (concentration) of said two or more peptides as compared to a reference amount for said two or more peptides is indicative of the subject having Alzheimer's disease; or
(c) wherein a change in amount (concentration) of said two or more peptides as compared to a reference amount for said two or more peptides is indicative of the subject having Alzheimer's disease.

In some cases of the method of this aspect of the invention, a change in amount of the two or more biomarkers is indicative of said subject having rapidly progressing AD, more severe cognitive impairment and/or more severe brain pathology.

The method according to this and other aspects of the invention may comprise comparing said amount of the two or more peptides with a reference level. In light of the present disclosure, the skilled person is readily able to determine a suitable reference level, e.g. by deriving a mean and range of values from samples derived from a population of subjects. In some cases, the method of this and other aspects of the invention may further comprise determining a reference level above which the amount of the two or more peptides can be considered to indicate an aggressive form of AD and/or a poor prognosis, particularly rapidly progressing AD, more severe cognitive impairment and/or more severe brain pathology. However, the reference level is preferably a pre-determined value, which may for example be provided in the form of an accessible data record. The reference level may be chosen as a level that discriminates more aggressive AD from less aggressive AD, particularly a level that discriminates rapidly progressing AD (e.g. a decline in a mini-mental state examination (MMSE) score of said subject at a rate of at least 2 MMSE points per year; and/or a decline in an AD assessment scale—cognitive (ADAS-Cog) score of said subject at a rate of at least 2 ADAS-Cog points per year) from non-rapidly progressing AD (e.g. a decline in an MMSE score of said subject at a rate of not more than 2 MMSE points per year; and/or a decline in an ADAS-Cog score of said subject at a rate of not more than 2 ADAS-Cog points per year). Preferably, the reference level is a value expressed as a concentration of each of said two or more peptides in units of mass per unit volume of a liquid sample or unit mass of a tissue sample.

In accordance with the method of this and other aspects of the invention, the biological sample may comprise blood plasma, blood cells, serum, saliva, cerebro-spinal fluid (CSF) or a tissue biopsy. Preferably, the biological sample has previously been isolated or obtained from the subject. The biological sample may have been stored and/or processed (e.g. to remove cellular debris or contaminants) prior to determining the amount (e.g. concentration) of the two or more peptides in the sample. However, in some cases the method may further comprise a step of obtaining the biological sample from the subject and optionally storing and/or processing the sample prior to determining the amount (e.g. concentration) of the two or more peptides in the sample. Preferably, the biological sample comprises blood plasma and the method comprises quantifying the blood plasma concentration of the two or more peptides.

In a preferred embodiment, the amount of the two or more biomarkers in the sample may be enriched prior to determination by specific antibodies. Such methods are well-known in the art.

In some cases the reference level may be chosen according to the assay used to determine the amount of the two or more peptides. A reference level in this range may represent a threshold dividing subjects into those below who are more likely to have a less aggressive form of AD (e.g. non-rapidly progressing AD) from those above who are more likely to have a more aggressive form of AD (e.g. rapidly progressing AD). However, the reference level may be a value that is typical of a less aggressive form of AD (e.g. non-rapidly progressing AD), in which case a subject having a reading significantly above the reference level may be considered as having or probably having an aggressive form of AD (e.g. rapidly progressing AD). Whereas the reference level may be a value that is typical of a more aggressive form of AD (e.g. rapidly progressing AD), in which case a subject having a reading significantly below the reference level may be considered as having or probably having a less aggressive form of AD (e.g. non-rapidly progressing AD).

In accordance with the method of this and other aspects of the invention, the method may further comprise determining one or more additional indicators of risk of AD, severity of AD, course of AD (such as rate or extent of AD progression). Such additional indicators may include one or more (such as 2, 3, 4, 5 or more) indicators selected from: brain imaging results (including serial structural MRI), cognitive assessment tests (including MMSE or ADAS-Cog), APOE4 status (particularly presence of one or more APOE4 ε4 alleles), fibrillar amyloid burden (particularly fibrillar amyloid load in the entorhinal cortex and/or hippocampus), CSF levels of Aβ and/or tau, presence of mutation in an APP gene, presence of mutation in a presenilin gene and presence of mutation in a clusterin gene. In some cases the method in accordance with this and other aspects of the invention is used as part of a panel of assessments for diagnosis, prognosis and/or treatment monitoring in a subject having or suspected of having AD.

In accordance with the method of this and other aspects of the invention, determining the amount of the two or more biomarker peptides in the biological sample may be achieved using any suitable method. The determination may involve direct quantification of the two or more peptides mass or concentration. The determination may involve indirect quantification, e.g. using an assay that provides a measure that is correlated with the amount (e.g. concentration) of the two or more peptides. In certain cases of the method of this and other aspects of the invention, determining the amount of the two or more peptide biomarkers comprises:
contacting said sample with specific binding members that selectively and independently bind to the two or more peptides; and
detecting and/or quantifying a complex formed by said specific binding members and the two or more peptides.

The specific binding member may be an antibody or antibody fragment that selectively binds to the peptide biomarker. For example, a convenient assay format for determination of a peptide concentration is an ELISA. The determination may comprise preparing a standard curve using standards of known for the peptide concentration and comparing the reading obtained with the sample from the subject with the standard curve thereby to derive a measure of the peptide biomarker concentration in the sample from the subject. A variety of methods may suitably be employed for determination of peptide amount (e.g. concentration), non-limiting examples of which are: Western blot, ELISA (Enzyme-Linked Immunosorbent assay), RIA (Radioimmunoassay), Competitive EIA (Competitive Enzyme Immunoassay), DAS-ELISA (Double Antibody Sandwich-ELISA), liquid immunoarray technology (e.g. Luminex xMAP technology or Becton-Dickinson FACS technology), immunocytochemical or immunohistochemical techniques, techniques based on the use protein microarrays that include specific antibodies, “dipstick” assays, affinity chromatography techniques and ligand binding assays. The specific binding member may be an antibody or antibody fragment that selectively binds a peptide biomarker. Any suitable antibody format may be employed, as described further herein. A further class of specific binding members contemplated herein in accordance with any aspect of the present invention comprises aptamers (including nucleic acid aptamers and peptide aptamers). Advantageously, an aptamer directed to the peptide biomarker may be provided using a technique such as that known as SELEX (Systematic Evolution of Ligands by Exponential Enrichment), described in U.S. Pat. Nos. 5,475,096 and 5,270,163.

In some cases of the method in accordance with this and other aspects of the invention, the determination of the amount of the peptide biomarkers comprises measuring the level of peptide by mass spectrometry. Techniques suitable for measuring the level of a peptides by mass spectrometry are readily available to the skilled person and include techniques related to Selected Reaction Monitoring (SRM) and Multiple Reaction Monitoring (MRM)isotope dilution mass spectrometry including SILAC, AQUA (as disclosed in WO 03/016861; the entire contents of which is specifically incorporated herein by reference) and TMTcalibrator (as disclosed in WO 2008/110581; the entire contents of which is specifically incorporated herein by reference). WO 2008/110581 discloses a method using isobaric mass tags to label separate aliquots of all proteins in a reference plasma sample which can, after labelling, be mixed in quantitative ratios to deliver a standard calibration curve. A patient sample is then labelled with a further independent member of the same set of isobaric mass tags and mixed with the calibration curve. This mixture is then subjected to tandem mass spectrometry and peptides derived from specific proteins can be identified and quantified based on the appearance of unique mass reporter ions released from the isobaric mass tags in the MS/MS spectrum.

By way of a reference level, the biomarker peptides as selected from Tables, 2, 3 and 4 may be used. In some cases, when employing mass spectrometry based determination of protein markers, the methods of the invention comprises providing a calibration sample comprising at least two different aliquots comprising the biomarker peptide, each aliquot being of known quantity and wherein said biological sample and each of said aliquots are differentially labelled with one or more isobaric mass labels. Preferably, the isobaric mass labels each comprise a different mass spectrometrically distinct mass marker group.

Accordingly, in a preferred embodiment of the invention, the method comprises determining the presence or expression level of two or more of the marker proteins selected from Table 2 by Selected Reaction Monitoring using one or more determined transitions for the known protein marker derived peptides as provided in Table 3 or Table 4; comparing the peptide levels in the sample under test with peptide levels previously determined to represent AD, MCI or normal; and determining the form or stage of dementia, e.g. AD or MCI based on changes in expression of said two or more marker proteins. The comparison step may include determining the amount of the biomarker peptides from the sample under test with known amounts of corresponding synthetic peptides. The synthetic peptides are identical in sequence to the peptides obtained from the sample, but may be distinguished by a label such as a tag of a different mass or a heavy isotope.

One or more of these synthetic biomarker peptides (with or without label) as identified in Tables 2, 3 or 4 form a further aspect of the present invention. These synthetic peptides may be provided in the form of a kit for the purpose of diagnosing AD or MCI in a subject.

Other suitable methods for determining levels of protein expression include surface-enhanced laser desorption ionization-time of flight (SELDI-TOF) mass spectrometry; matrix assisted laser desorption ionization-time of flight (MALDI-TOF) mass spectrometry, including LS/MS/MS; electrospray ionization (ESI) mass spectrometry; as well as the preferred SRM and TMT-SRM.

In a further aspect of the invention, there is provided a kit for use in carrying out the methods described above, in particular diagnosing AD or MCI in a sample obtained from a subject.

In all embodiments, the kit allows the user to determine the presence or level of expression of a plurality of analytes selected from a plurality of marker proteins or fragments thereof provided in Table 2, Table 3 or Table 4; antibodies against said marker proteins and nucleic acid molecules encoding said marker proteins or a fragments thereof, in a sample under test; the kit comprising
(a) a solid support having a plurality of binding members, each being independently specific for one of said plurality of analytes immobilised thereon;
(b) a developing agent comprising a label; and, optionally
(c) one or more components selected from the group consisting of washing solutions, diluents and buffers.

The binding members may be as described above.

In one embodiment, the kit may provide the analyte in an assay-compatible format. As mentioned above, various assays are known in the art for determining the presence or amount of a protein, antibody or nucleic acid molecule in a sample. Various suitable assays are described below in more detail and each form embodiments of the invention.

The kit may additionally provide a standard or reference which provides a quantitative measure by which determination of an expression level of one or more marker proteins can be compared. The standard may indicate the levels of the two or more biomarkers which indicate AD or MCI

The kit may also comprise printed instructions for performing the method.

In a preferred embodiment, the kit may be for performance of a mass spectrometry assay and may comprise a set of reference peptides as set out in Table 2, Table 3 or Table 4 (e.g. SRM peptides) [specific combinations of said peptides can be found in FIG. 5 or FIG. 7] (e.g. SRM peptides) in an assay compatible format wherein each peptide in the set is uniquely representative of each of the plurality of marker proteins. Preferably two and more preferably three such unique peptides are used for each protein for which the kit is designed, and wherein each set of unique peptides are provided in known amounts which reflect the levels of such proteins in a standard preparation of said sample. Optionally the kit may also provide protocols and reagents for the isolation and extraction of proteins from said sample, a purified preparation of a proteolytic enzyme such as trypsin and a detailed protocol of the method including details of the precursor mass and specific transitions to be monitored. The peptides may be synthetic peptides and may comprise one or more heavy isotopes of carbon, nitrogen, oxygen and/or hydrogen.

In all aspects of the invention, the two or more peptides which make up the biomarker panel are selected from Table 2, Table 3 or Table 4. In preferred embodiments, three or more, four or more, five or more, or six or more peptides make up the biomarker panel.

In all aspects of the invention, the peptide biomarker may comprise or consist of the peptide selected from Tables 2, 3 or 4. Where the peptide biomarker comprises the selected sequence provided in Tables 2, 3 or 4, it is preferable that it is no more than 50 amino acids in length, more preferably no more than 45, 40, 35 or 30 amino acids in length. In some embodiments, the biomarker peptide may comprise a peptide which differs from the peptide selected from Table 2, 3 or 4 by no more than one, two, three, four, five or six amino acids.

In particular, the inventors have determined based on mathematical modelling specific combinations of peptides which when combined provide a biomarker panel having greater specificity for AD or MCI respectively.

Accordingly, for all aspects of the present invention, the two or more peptides preferably comprises the combination of peptides selected from the group consisting of Y1 to Y30 in FIG. 5 or selected from the group consisting of Y1 to Y30 in FIG. 7.

In a further preferred embodiment, the two or more biomarker peptides are:—

For diagnosis AD

### Y1=VYAYYNJEESCTR*p1+TAGWNJPMGJJYNK*p2+SSSKDNJR*p3+DSSVPNTGTAR*p4

With the fitted parameters p1=−0.575035, p2=0.331443, p3=−0.319553, p4=0.0720402

The sensitivity of this model is 0.42 and the specificity is 0.98.—See FIG. 3

For Diagnosing MCI

### Y1=EFN_AETFTFHADICTISEK*p1+QGIPFFGQVR*p2−TEGDGVYTINDK*p3+NTCNHDEDTWVECEDPFDIR*p4+SSSKDNIR*p5−NIIDRQDPPSVVVTSHQAPGEK*p6

With the fitted parameters p1=0.345556, p2=0.281846, p3=0.138583, p4=0.193817, p5=0.222568, p6=0.222843 The sensitivity of this model is 0.71 and the specificity is 0.95—See FIG. 4

The algorithm (as shown in FIG. 1) used computes a total score. If the total is >0.5 it is in the specific disease class (i.e. AD or MCI depending on the model) whilst <0.5 is in the other classes (i.e. MCI and control or AD and control depending on the model). Accordingly, in a preferred embodiment score are computed in line with the GMDH algorithm which then sets the threshold value.

Certain aspects and embodiments of the invention will now be illustrated by way of example and with reference to the figures and tables described above. The present invention includes the combination of the aspects and preferred features described except where such a combination is clearly impermissible or is stated to be expressly avoided. All documents mentioned in this specification are incorporated herein by reference in their entirety for all purposes.

### BRIEF DESCRIPTION OF THE FIGURES

FIG. 1: Polynomial model used after GMDH modeling

FIG. 2: Selection of plasma samples based on a balanced design

FIG. 3: Prediction of the patients to belong to the group of AD patients or to the joint group of MCI+Control cases based on the computed functional value Y1 of the model. If Y1 exceeds 0.5 the patient is subjected to the AD group.

FIG. 4: Prediction of the patients to belong to the group of MCI patients or to the joint group of AD+Control cases based on the computed functional value Y1 of the model. If Y1 exceeds 0.5 the patient is subjected to the AD group.

FIG. 5: Top 30 AD model equations selected by the GMDH algorithm to predict AD versus (MC+controls)

FIG. 6: GMDH criterion of the top 30 AD versus (MCI+Control) models defined by 1-model coverage.

FIG. 7: Top 30 MCI model equations selected by the GMDH algorithm to predict MCI versus (AD+controls)

FIG. 8: GMDH criterion of the top 30 MCI versus (AD+Control) models defined by 1-model coverage.

FIG. 9: Contour diagram using the peptide SJFTDJEAENDVJHCVAFAVPK (x-Axis) and JFJEPTRK (Y-Axis). The density of patients in this two dimensional space is depicted by colour from sparse (blue) to dense (orange).

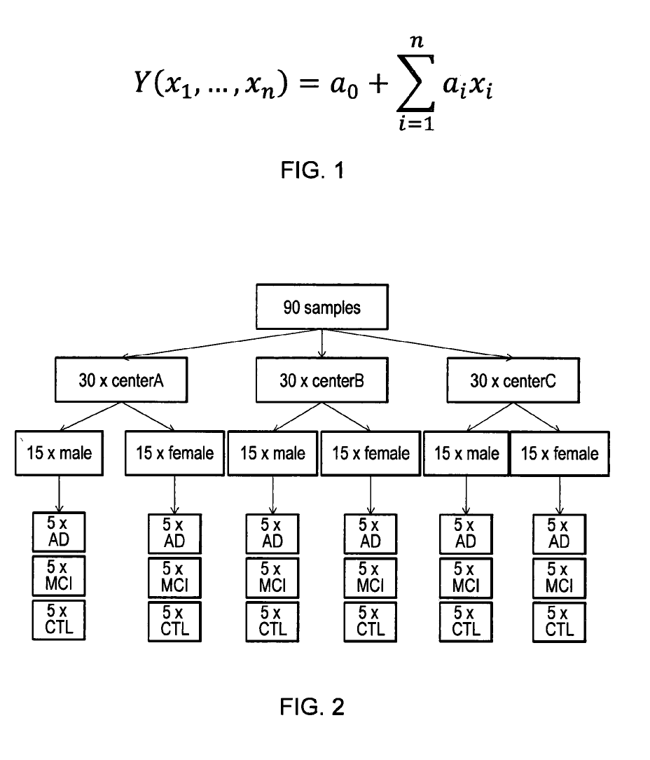

### DETAILED DESCRIPTION

Liquid chromatography—mass spectrometry (LC-MS/MS) based proteomics has proven to be superior over conventional biochemical methods at identifying and precisely quantifying thousands of proteins from complex samples including cultured cells (prokaryotes/eukaryotes), and tissue (Fresh Frozen/formalin fixed paraffin embedded), leading to the identification of novel biomarkers in an unbiased manner [7, 8, 9]. The present inventors have not only identified such novel biomarkers, but have determined combinations of specific peptides which have greater predictive power and therefore lead to more accurate diagnosis of the forms of dementia and in particular the distinction between AD and MCI.

The degree to which expression of a biomarker differs between AD and MCI, need only be large enough to be visualised via standard characterisation techniques, such as silver staining of 2D-electrophoretic gels. Other such standard characterisation techniques by which expression differences may be visualised are well known to those skilled in the art. These include successive chromatographic separations of fractions and comparisons of the peaks, capillary electrophoresis, separations using micro-channel networks, including on a micro-chip, SELDI analysis and isobaric and isotopic Tandem Mass Tag analysis.

Chromatographic separations can be carried out by high performance liquid chromatography as described in Pharmacia literature, the chromatogram being obtained in the form of a plot of absorbance of light at 280 nm against time of separation. The material giving incompletely resolved peaks is then re-chromatographed and so on.

Capillary electrophoresis is a technique described in many publications, for example in the literature “Total CE Solutions” supplied by Beckman with their P/ACE 5000 system. The technique depends on applying an electric potential across the sample contained in a small capillary tube. The tube has a charged surface, such as negatively charged silicate glass. Oppositely charged ions (in this instance, positive ions) are attracted to the surface and then migrate to the appropriate electrode of the same polarity as the surface (in this instance, the cathode). In this electroosmotic flow (EOF) of the sample, the positive ions move fastest, followed by uncharged material and negatively charged ions. Thus, proteins are separated essentially according to charge on them.

Micro-channel networks function somewhat like capillaries and can be formed by photoablation of a polymeric material. In this technique, a UV laser is used to generate high energy light pulses that are fired in bursts onto polymers having suitable UV absorption characteristics, for example polyethylene terephthalate or polycarbonate. The incident photons break chemical bonds with a confined space, leading to a rise in internal pressure, mini-explosions and ejection of the ablated material, leaving behind voids which form micro-channels. The micro-channel material achieves a separation based on EOF, as for capillary electrophoresis. It is adaptable to micro-chip form, each chip having its own sample injector, separation column and electrochemical detector: see J. S. Rossier et al., 1999, Electrophoresis 20: pages 727-731. Surface enhanced laser desorption ionisation time of flight mass spectrometry (SELDI-TOF-MS) combined with ProteinChip technology can also provide a rapid and sensitive means of profiling proteins and is used as an alternative to 2D gel electrophoresis in a complementary fashion. The ProteinChip system consists of aluminium chips to which protein samples can be selectively bound on the surface chemistry of the chip (eg. anionic, cationic, hydrophobic, hydrophilic etc). Bound proteins are then co-crystallised with a molar excess of small energy-absorbing molecules. The chip is then analysed by short intense pulses of N2 320 nm UV laser with protein separation and detection being by time of flight mass spectrometry. Spectral profiles of each group within an experiment are compared and any peaks of interest can be further analysed using techniques as described below to establish the identity of the protein.

Isotopic or isobaric Tandem Mass Tags® (TMT®) (Thermo Scientific, Rockford, USA) technology may also be used to detect differentially expressed proteins which are members of a biomarker panel described herein. Briefly, the proteins in the samples for comparison are optionally digested, labelled with a stable isotope tag and quantified by mass spectrometry. In this way, expression of equivalent proteins in the different samples can be compared directly by comparing the intensities of their respective isotopic peaks or of reporter ions released from the TMT reagents during fragmentation in a tandem mass spectrometry experiment.

Unless context dictates otherwise, the descriptions and definitions of the features set out above are not limited to any particular aspect or embodiment of the invention and apply equally to all aspects and embodiments which are described.

Thus, the features set out above are disclosed in all combinations and permutations.

### EXPERIMENTAL

In the present specification amino acid residues within peptide sequences are denoted using the IUPAC single letter code convention. In cases where residue identification between isoleucine and leucine is ambiguous the single letter code ‘J’ is used.

Proteins are typically identified herein by reference to their Uniprot Accession Number or Uniprot ID. It is understood in the art that this reference relates to the annotated amino acid sequence ascribed to the Uniprot Accession Number at the date of filing. Since Uniprot provides a full history of sequence additions and amendments within the page for each protein it is possible for the skilled practitioner to identify the protein referred to within this specification without undue burden.

In these experiments a set of 90 samples have been labelled with isotopic TMT reagents (heavy and light) and analysed for peptide analytes by means of mass spectrometric analysis using an LTQ Orbitrap Velos (Thermo Scientific, Germany) using a hybrid Inclusion List/Data Dependent Acquisition Strategy.

Data is then further analysed in term of identification and quantifications. Finally, this data was statistically analysed using a mixed effect model including relevant covariates for regulated peptides and proteins in Alzheimer disease (AD) and Mild cognitive impairment (MCI). In addition, polynomial regression models were computed to combine a set of markers together to achieve a biomarker panel with increased sensitivity and specificity.

The samples have been labelled and processed using isotopic TMT0 and TMT6(127) reagents, which exhibit a 5 Dalton mass difference, alkylated and trypsinated. To each of the samples a TMT6 (heavy) labelled reference material was added containing a mixture of all samples. The samples have been processed by means of Maxquant and the peptide intensities were exported and statistically processed. MaxQuant exported a highly reproducible quantitative data matrix which is supposed to depend on the retention time/mass alignment done by the analysis software.

A set of 31 significantly peptide markers were found in the univariate statistical modelling to be useful for the analysis of AD and MCI. For the panel discovery a set of 30 most relevant peptide marker constituents was compiled for three models a 4 parametric AD model, a 2 parametric AD model, a 4 parametric MCI model and a 6 parametric MCI model. Out of these marker lists polynomial models can be formed.

In each model a composite score ‘Y’ is computed based on the relative abundance of each panel member peptide relative to a universal reference control plasma. An increased value of Y relates to the likelihood of AD or MCI in the respective model.

### Example 1

### Sample Preparation of Plasma Samples for the Subsequent Measurement with an Isotopic Mass Spectrometry Based Workflow

90 plasma samples have been prepared according to a standard operating protocol. Per sample, a plasma volume of 1.25 μL has been processed. In brief, defined volumes of the samples have been diluted by a two-step procedure, and then subjected to reduction, alkylation and digestion with trypsin. The tryptic peptides were then labelled with TMTzero reagent and purified using strong cation exchange (SCX) cartridges according to a standard operating procedure. Following purification, the samples have been transferred to microtiter plates, whereby three aliquots have been taken from each sample. Per plate position, a plasma volume equivalent of 0.375 μL has been charged.

In detail, crude human plasma samples have been diluted by factor 80 with dilution buffer (100 mM TEAB pH 8.5 and 0.1% SDS). Per diluted plasma sample, 100 μL containing 1.25 μL plasma equivalent volume was used for further processing. Proteins have been reduced with TCEP (1 mM final concentration, 1 h, 55° C.) and alkylated with iodoacetamide (7.5 mM final concentration, 1 h, room temperature). Subsequently, the protein samples were digested with trypsin (addition of 20 μL of a 0.4 μg/μL stock solution) by overnight incubation at 37° C. The digested plasma samples were then labeled with the TMTzero reagent (addition of 40 μL of 60 mM stock solution in acetonitrile) by 1 h incubation at room temperature. Then, 8 μL of an aqueous hydroxylamine solution (5%) have been added to quench excess of labeling reagent.

The processed samples have been purified with SCX cartridges (self-packed cartridges using SP Sepharose Fast Flow, Sigma). After addition of 3 mL 50% acetonitrile with 0.1% TFA, samples have been loaded onto the cartridge and washed with 4 mL 50% acetonitrile with 0.1% TFA. Then, the samples have been eluted with 1.5 mL of 400 mM ammonium acetate in 25% acetonitrile. Finally, the samples have been dried in a vacuum concentrator.

### Preparation of a Reference Sample

A reference sample has been obtained by mixing of 100 different individual plasma samples after 80 fold dilution as described above. 300 μL of this mixed reference sample, containing a plasma equivalent volume of 3.75 μL, have been used for further processing. Proteins have been reduced with TCEP (1 mM final concentration, 1 h, 55° C.) and alkylated with iodoacetamide (7.5 mM final concentration, 1 h, room temperature). Subsequently, the protein samples were digested with trypsin (addition of 60 μL of a 0.4 μg/μL stock solution) by overnight incubation at 37° C. The digested plasma samples were then labeled with the TMT⁶-127 reagent (addition of 120 μL of 60 mM stock solution in acetonitrile) by 1 h incubation at room temperature. Then, 24 μL of an aqueous hydroxylamine solution (5%) have been added to quench excess of labeling reagent.

The processed reference sample has been aliquoted into 3 equal portions; each aliquot has been purified with SCX cartridges as given above. After addition of 3 mL 50% acetonitrile with 0.1% TFA, the aliquots have been loaded onto the cartridge and washed with 4 mL 50% acetonitrile with 0.1% TFA. Then, the aliquots have been eluted with 1.5 mL of 400 mM ammonium acetate in 25% acetonitrile. Finally, the aliquots were re-combined and the sample has been dried in a vacuum concentrator.

### Example 2

### Mass Spectrometric Analysis of Plasma Samples for the Purpose of Utilising an Isotopic Workflow

The lyophilised peptides from each sample and the reference prepared in example 1 were individually re-suspended in 2% ACN, 0.1% FA. Prior to mass spectrometry analysis an equal volume of each individual sample digest was mixed with the reference sample digest producing 90 analytical isotopic samples. Each analytical isotopic sample was injected onto a 0.1×20 mm column packed with ReproSil C18, 5 μm (Dr. Maisch), using the Thermo Scientific Proxeon EASY-nLC II system. Peptides were then resolved using an increasing gradient of 0.1% formic acid in acetonitirile (5 to 30% over 90 min) through a 0.075×150 mm self-packed column with ReproSil C18, 3 μm (Dr. Maisch) at a flow rate of 300 nL/min. Mass spectra were acquired on a Thermo Scientific LTQ Orbitrap Velos throughout the chromatographic run (115 minutes), using 10 higher collision induced dissociation (HCD) FTMS scans at 7,500 resolving power @ 400 m/z, following each FTMS scan (30,000 resolving power @ 400 m/z). HCD was carried out on a time-dependent inclusion list containing 115 peptides with a mass accuracy window of ±25 ppm.

This list of selected peptides was focussed on the following proteins:

TABLE 1
AD SRM proteins and number of peptides included
in the LTQ Orbitrap Velos method

| Protein | Number of peptides |
|---|---|
| Alpha-2-macroglobulin | 15 |
| Apolipoprotein E | 13 |
| Complement C3 | 14 |
| Complement factor H | 10 |
| Gelsolin | 12 |
| Clusterin | 11 |
| Fibrinogen gamma chain | 12 |
| Serum amyloid P-component | 8 |
| Serotransferrin | 5 |
| Alpha-1-antitrypsin | 5 |
| Alpha-2-HS-glycoprotein | 5 |
| Serum albumin | 5 |

If none of the peptides in the inclusion list could be detected in MS1, the remaining precursors of the 10 most intense precursors are selected for HCD fragmentation. Precursors already selected from each FTMS scan were then put on a dynamic exclusion list for 30 secs (25 ppm m/z window). AGC ion injection target for each FTMS1 scan were 1,000,000 (500 ms max injection time). AGC ion injection target for each HCD FTMS2 scan were 50,000 (500 ms max ion injection time, 2μscans. A peptide expression matrix was assembled using the software Maxquant importing all available mass spectrometry runs and assembling all relevant intensity (pair) values of the heavy and light labelled peptides. Peptides were also searched using Maxquant.

In total 199 protein groups have been identified, represented by 2089 distinct peptides.

### Example 3

### Creation of a Univariate Statistical Model Using Mixed Effect Modelling (GLM)

Mixed effect modelling allows for the selection and prioritization of biomarkers according to their statistical relevance. It allows one to include relevant covariates into the models to separate the variance, which was mainly driven by the covariates from the information related to the diagnosis. The models used were using the information of the disease class, study centre, where the samples were collected, gender, age and storage time of the samples a relevant in the model.

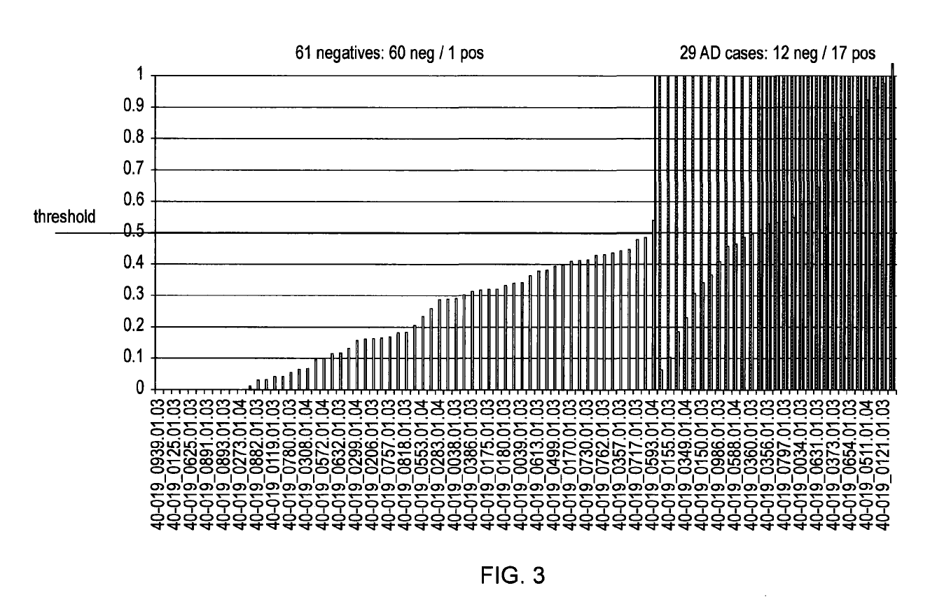

The samples used belong to different selected groups balanced for some parameters in the experimental design: See FIG. 3.

In total 199 protein groups have been identified, represented by 2089 distinct peptides. The expression matrix was filtered to remove peptide measurements which contained less than 70% of available datapoints contain at least 70%

Thereof 152 proteins groups and 1630 peptides was considered during univariate statistical analysis. The expression matrix was filtered where the quantitative expression matrix contained at least for 70% of the available samples quantitative.

A linear mixed effect model was computed using the peptide data. For all computation R version 2.13 was used. For the linear mixed effect model the following factors were used:
Diagnosis (three levels)
AD, MCI, CTL
APOE (6 different allelic geneotypes)
2/2, 2/3, 2/4, 3/3, 3/4, 4/4
Centre (three different sample collection centers)
2, 4, 5
Gender (two levels)
Female, Male
Continous covariates
   Age (patient age)
   Age_samples (storage time of samples in the freezer)

Peptides with significant value less than p<0.05 were considered relevant in the univariate model.

At the peptide level, 31 entities appeared to be relevant as shown in Table 2 below.

TABLE 2
Peptides with statistical significance
(LME p-value < 0.05) for the diagnosis AD

| | | | LME p- | LME p- |
|---|---|---|---|---|
| | | | value | value |
| | Accession | | diagnos | diagnos |
| Peptide sequence | number | Protein name | is AD | is MCI |
| FYSEKECR | P02760 | Protein AMBP | 0.011 | 0.444 |
| MFJSFPTTK | P69905 | Hemoglobin subunit | 0.011 | 0.598 |
| | | alpha | | |
| JGMFNJQHCK | P01009 | Alpha-1-antitrypsin | 0.012 | 0.022 |
| EGKQVGSGVTTDQVQAEAK | P01871-2 | Isoform 2 of Ig mu | 0.012 | 0.056 |
| | | chain C region | | |
| JAYGTQGSSGYSJR | H0YAC1 | Kallikrein B, plasma | 0.014 | 0.228 |
| | | (Fletcher factor) 1 | | |
| | | (Fragment) | | |
| TQVNTQAEQJRR | P06727 | Apolipoprotein A-IV | 0.019 | 0.368 |
| JVSANR | P01008 | Antithrombin-III | 0.019 | 0.312 |
| JSJTGTYDJKSVJGQJGJT | P01009 | Alpha-1-antitrypsin | 0.020 | 0.613 |
| K | | | | |
| FMQAVTGWK | P01019 | Angiotensinogen | 0.020 | 0.006 |
| YGJVTYATYPK | B4E1Z4 | Complement factor B | 0.022 | 0.053 |
| VRVEJJHNPAFCSJATTK | P01024 | Complement C3 | 0.023 | 0.010 |
| HJEVDVWVJEPQGJR | P19823 | Inter-alpha-trypsin | 0.023 | 0.024 |
| | | inhibitor heavy | | |
| | | chain H2 | | |
| SFFPENWJWR | B0UZ83 | Complement component | 0.025 | 0.874 |
| | | 4A (Rodgers blood | | |
| | | group) | | |
| REQPGVYTK | H0YAC1 | Kallikrein B, plasma | 0.025 | 0.499 |
| | | (Fletcher factor) 1 | | |
| | | (Fragment) | | |
| TJPEPCHSK | H0YAC1 | Kallikrein B, plasma | 0.026 | 0.003 |
| | | (Fletcher factor) 1 | | |
| | | (Fragment) | | |
| JGMFNJQHCKK | P01009 | Alpha-1-antitrypsin | 0.027 | 0.388 |
| NJAVSQVVHK | G3V5I3 | Serpin peptidase | 0.028 | 0.670 |
| | | inhibitor, Glade A | | |
| | | (Alpha-1 | | |
| | | antiproteinase, | | |
| | | antitrypsin), member | | |
| | | 3, isoform CRA_b | | |
| QGPVNJJSDPEQGVEVTGQ | B7ZKJ8 | ITIH4 protein | 0.029 | 0.047 |
| YER | | | | |
| SJGECCDVEDSTTCFNAK | D6RAK8 | Group-specific | 0.030 | 0.656 |
| | | component (vitamin | | |
| | | D-binding protein) | | |
| QVQJVQSGGGJVKPGGSJR | P01762 | Ig heavy chain V-III | 0.033 | 0.071 |
| | | region TRO | | |
| DQGHGHQR | P01042 | Kininogen-1 | 0.034 | 0.148 |
| SHKWDREJJSER | P02790 | Hemopexin | 0.038 | 0.618 |
| JTJJSAJVETR | G3V5I3 | Serpin peptidase | 0.039 | 0.628 |
| | | inhibitor, clade A | | |
| | | (Alpha-1 | | |
| | | antiproteinase, | | |
| | | antitrypsin), member | | |
| | | 3, isoform CRA_b | | |
| YYTYJJMNK | P01024 | Complement C3 | 0.040 | 0.053 |
| DQJTCNKFDJK | P01024 | Complement C3 | 0.041 | 0.002 |
| SVJGQJGJTK | P01009 | Alpha-1-antitrypsin | 0.043 | 0.903 |
| SJTSCJDSK | O95445 | Apolipoprotein M | 0.044 | 0.536 |
| EKGYPK | P02790 | Hemopexin | 0.044 | 0.793 |
| VRESDEETQJK | P04114 | Apolipoprotein B-100 | 0.044 | 0.138 |
| EJJSVDCSTNNPSQAK | P10909-2 | Isoform 2 of | 0.045 | 0.282 |
| | | Clusterin | | |
| HPYFYAPEJJFFAKR | CON_P027 | Serum albumin | 0.048 | 0.653 |
| | 68-1 | | | |

### Example 4

### Creation of a Multimarker Model Using GMDH (Group Modelling and Data Handling)

The inventors have discovered over 30 peptides with statistically significant differences in blood plasma levels in patients with AD or MCI relative to controls. However, the diagnostic utility of individual biomarkers is generally improved when used in combination. Thus to enhance the quality of predictions using biomarkers it is possible to combine a set of multiple markers in a model. For this purpose a polynomial regression model was created using the GMDH (group modelling and data handling) algorithm. GMDH is family of inductive algorithms for computer-based mathematical modelling of multi-parametric datasets that features fully automatic structural and parametric optimization of models which delivers simple but highly reliable polynomial models using a data driven (inductive) approach.

In the present case a simple regression models with no higher order terms was used:

To compute the GMDH models the software GMDH Shell 3.8 (http://www.gmdhshell.com/) was used. The data matrix used contained expression values for 1104 peptides and the log 2 transformed expression values for 90 samples. The expression matrix (see example 1) was filtered so that at least 80% of variables were present.

GMDH shell creates a set of alternative polynomial models, which are ranked according to their predictive utility in a top down fashion. The program settings used as cross validation (9 folds), and variable preselection (only the top 200 relevant variables were used). The model complexity was selected to be fixed 4 parameters (variables). Two models were computed to predict AD (Alzheimer's) versus MCI (mild cognitive impairment) plus control samples, and alternatively MCI versus the joint group of AD plus control samples.
“Model AD” AD˜(MCI+controls)
“Model MCI” MCI˜(AD+controls)

The linear model shall be interpreted in the following way: If the computed value y exceeds the threshold 0.5 than the case belongs to the class (either AD for “model AD” or MCI for “model MCI” depending on the model). If the computed value is below the threshold the sample belongs to the alternative group (model 1: MCI/control or model 2: AD/control)

It is important to note that due to the use of MaxQuant mass spectrometry quantification software it is not possible to distinguish between the amino acids I or L, which are isotopic. Accordingly, where sequences are given from the MaxQuant analysis I and L are both replaced with the letter J.

The following tables indicate the different attributes, which were found to be relevant to compose 4 parametric models. The score is related to the number of times GMDH Shell was selecting a dedicated attribute in the set of best 200 models. Consequently, this table represents the most relevant variables, which predict the occurrence of Alzheimer's disease, or alternatively the presence of mild cognitive impairment MCI. Individual models can then be built from these variables to compose a linear equation.

Here, attributes with higher scores (score >15) are more likely to be included into the model either as first or second choice attribute complemented by any other attribute.

TABLE 3
Set of attributes used for 4 parametric
models and their usage statistics for
prediction of AD (Amino Acid code J
represents either isoleucine (I) or
leucine (L))

| Peptide | Usage | Uniprot_ID |
|---|---|---|
| JCMGSGJNJCEPNNK | 109 | P02787 |
| VKDJATVYVDVJKDSGR | 108 | P02647 |
| SSSKDNJR | 69 | P00450 |
| TAGWNJPMGJJYNK | 68 | P02787 |
| SEVAHR | 60 | P02768-1 |
| DSSVPNTGTAR | 46 | P01031 |
| EAVSGR | 29 | B7ZKJ8 |
| VYAYYNJEESCTR | 24 | P01024 |
| SJFTDJEAENDVJHCVAFAVPK | 23 | H0YGH4 |
| AGAFCJSEDAGJGJSSTASJR | 16 | H0YGH4 |
| JFJEPTRK | 15 | P00747 |
| SJDFTEJDVAAEKJDR | 12 | P01019 |
| HVVPNEVVVQR | 11 | P06396 |
| VEPJRAEJQEGAR | 11 | P02647 |
| RHPYFYAPEJJFFAK | 9 | P02768-1 |
| QHEKER | 8 | P02763 |
| TEGDGVYTJNDK | 7 | P00738 |
| DKCEPJEK | 6 | P02763 |
| DNCCJJDER | 6 | P02679 |
| DGYJFQJJR | 5 | P04196 |
| FYSEKECR | 4 | P02760 |
| GPTQEFK | 4 | H0YGH4 |
| JTJJSAJVETR | 4 | G3V5I3 |
| KCSTSSJJEACTFR | 4 | P02787 |
| MFJSFPTTK | 4 | P69905 |
| MPCAEDYJSVVJNQJCVJHEK | 4 | P02768-1 |
| TTVMVK | 4 | H0YGH4 |
| VFDEFKPJVEEPQNJJK | 4 | P02768-1 |

TABLE 4
Set of attributes used for 4 parametric
models and their usage statistics for
prediction of MCI (Amino Acid code J
represents either isoleucine (I) or
leucine (L))

| | | Uniprot |
|---|---|---|
| Peptide | count | ID |
| SSSKDNJR | 115 | P00450 |
| EFNAETFTFHADJCTJSEKER | 68 | P02768-1 |
| TEGDGVYTJNDK | 61 | P00738 |
| SGJSTGWTQJSK | 60 | P04217 |
| NTCNHDEDTWVECEDPFDJR | 42 | O43866 |
| SASDJTWDNJK | 37 | P02787 |
| VPQVSTPTJVEVSR | 34 | P02768-1 |
| AEFAEVSK | 32 | P02768-1 |
| RPSGJPER | 32 | P01715 |
| EJKEQQDSPGNKDFJQSJK | 21 | P08697 |
| HPDYSVVJJJR | 20 | P02768-1 |
| TPVSDRVTK | 19 | P02768-1 |
| NJREGTCPEAPTDECKPVK | 16 | P02787 |
| TEGDGVYTJNDKK | 16 | P00738 |
| NJJDRQDPPSVVVTSHQAPGEK | 15 | P25311 |
| DVFJGMFJYEYAR | 13 | P02768-1 |
| EFNAETFTFHADJCTJSEK | 13 | P02768-1 |
| JDAQASFJPK | 12 | P19827 |
| GNQESPK | 11 | P02751 |
| QGJPFFGQVR | 10 | H0YGH4 |
| JRTEGDGVYTJNDKK | 9 | P00738 |
| JSVJRPSK | 9 | B4E1Z4 |
| QSNNKYAASSYJSJTPEQWK | 8 | P0CG05 |
| DQFNJJVFSTEATQWRPSJVPASAENVNK | 7 | B7ZKJ8 |
| EVJJPK | 7 | P05546 |
| VGFYESDVMGR | 6 | H0YGH4 |
| RHPDYSVVJJJR | 5 | P02768-1 |
| VJVDHFGYTK | 5 | P04114 |
| DYFMPCPGR | 4 | P02790 |
| JJEJTGPK | 4 | P04217 |

### Example 5

### Investigating the Top Ranked Predictive Model for AD and MCI

### Designing an Optimum Panel for Diagnosis of AD

Using the GMDH scores calculated in Example 2 an optimum panel of four peptides was selected for the prediction of Alzheimer's disease. Across the 90 samples the model had a positive predictive value of 94.4% and a negative predictive value of 83.3%.

The four peptides were:

VYAYYNIEESCTR from human Complement C3 (Uniprot Acc. No. P01024);
TAGWNIPMGIIYNK from human serotransferrin (Uniprot Acc. No. P02787);
SSSKDNIR from human ceruloplasmin (Uniprot Acc. No. P00450); and
DSSVPNTGTAR from human Complement C5 (Uniprot Acc. No. P01031)

| | | Condition | |
|---|---|---|---|
| | Condition AD | Control/MCI | |
| model >0.5 | TP = 17 | FP = 1 | Positive predictive |
| | | | value = 0.944 |
| model <0.5 | FN = 12 | TN = 60 | Negative predictive |
| | | | value = 0.833 |
| | Sensitivity = 0.58 | Specificity = 0.98 | |

The linear equation for this panel is given below:

### Y1=[VYAYYNJEESCTR]*p1+[TAGWNJPMGJJYNK]*p2+[SSSKDNJR]*p3+[DSSVPNTGTAR]*p4

With the fitted parameters p1=−0.575035, p2=0.331443, p3=−0.319553, p4=0.0720402

The sensitivity of this model is 0.58 and the specificity is 0.98.—See FIG. 3

### Designing an Optimum Panel for MCI

Using the GMDH scores calculated in Example 2 an optimum panel of six peptides was selected for the prediction of Alzheimer's disease. Across the 90 samples the model had a positive predictive value of 88% and a negative predictive value of 86%.

The six peptides were:

EFN_AETFTFHADICTISEK from human serum albumin (Uniprot Acc. No. Q8IUK7);
QGIPFFGQVR from human alpha-2-macroglobulin (Uniprot Acc. No. P01023);
TEGDGVYTINDK from human haptoglobin (Uniprot Acc. No. P00739); NTCNHDEDTWVECEDPFDIR from human CD5 antigen-like protein (Uniprot Acc. No. 043866)
SSSKDNIR from human ceruloplasmin (Uniprot Acc. No. P00450); and
NIIDRQDPPSVVVTSHQAPGEK from human zinc-alpha-2-glycoprotein (Uniprot Acc. No. P25311)

The linear equation for this panel is given below

### Y1=[EFN_AETFTFHADICTISEK]*p1+[QGIPFFGQVR]*p2−[TEGDGVYTINDK]*p3+[NTCNHDEDTWVECEDPFDIR]*p4+[SSSKDNIR]*p5−[NIIDRQDPPSVVVISHQAPGEK]*p6

With the fitted parameters p1=0.345556, p2=0.281846, p3=0.138583, p4=0.193817, p5=0.222568, p6=0.222843

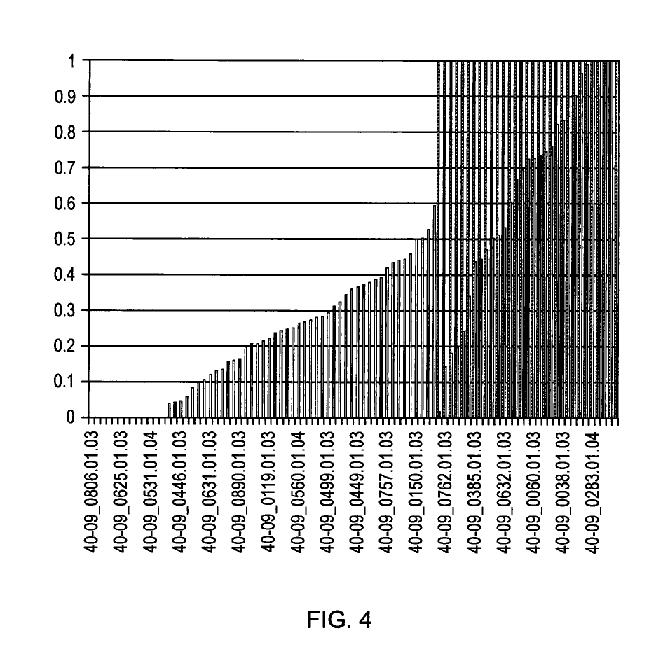

The sensitivity of the model was 0.71 and the specificity 0.95.—See FIG. 4

### Example 6

### Combination of a Set of 30 Best GMDH Models

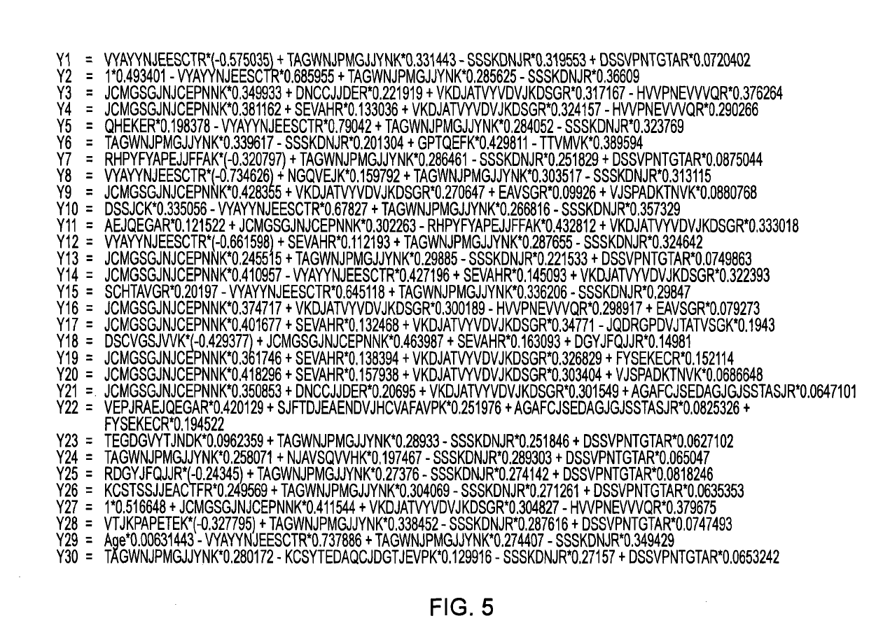

The GMDH algorithm produces a set of alternative models, which are suitable for the diagnosis of AD and MCI. This is achieved by maximizing the so called external criterion in the GMDH selection process. The best model appears as top ranked followed by a set of alternative models, which are ranked according to their utility. The top 30 models illustrate a preferable set of variables. The set of best 30 GMDH polynomial models including parameters fitted appears in FIG. 5 for the application AD versus (MCI+control)

The fitted parameters are related to the measurement process in the mass spectrometer. For a further implementation on other analytical procedures it is likely that they can differ. However, each equation selects a set of variables to be combined, which is related to the model structure (i.e. selection of the variables), which is the most relevant information present in these formulas. They describe preferable ways, which variables (measured peptides from which proteins) to combine out of the lists 3-5 to achieve the best models.

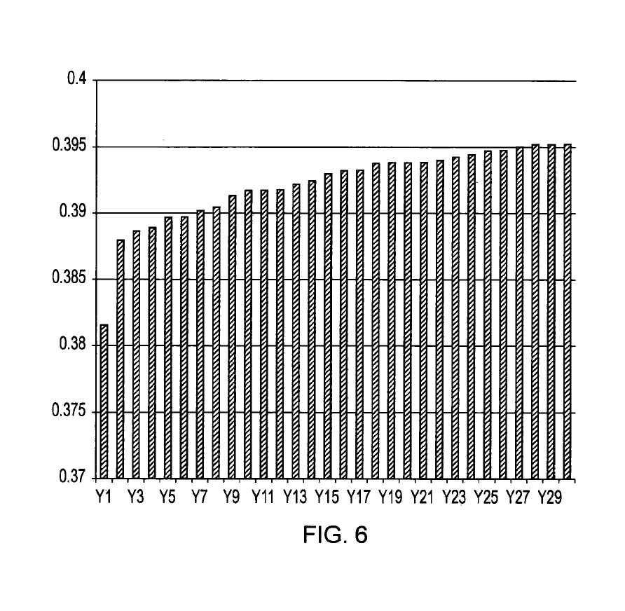

The graph of FIG. 6 indicates the GMDH criterion, which is related to the model quality, which is defined by 1-model coverage.

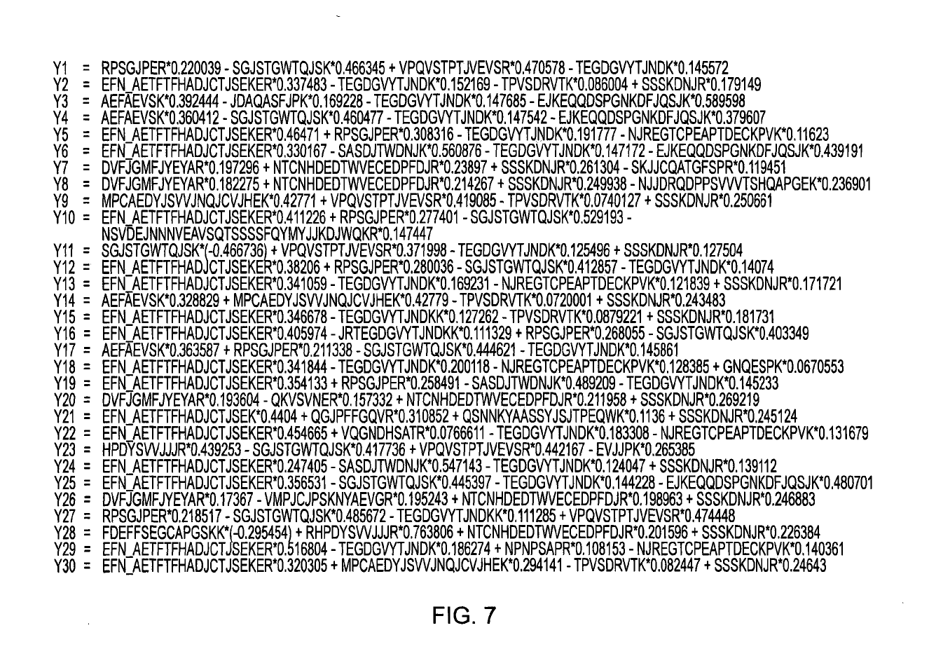

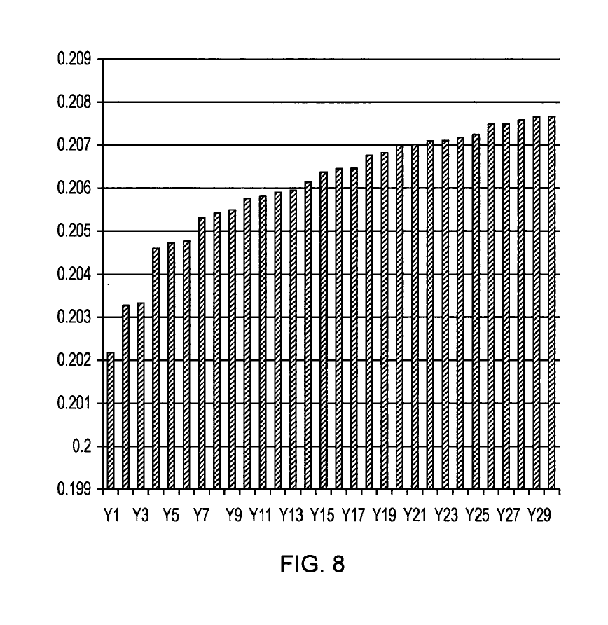

The table of FIG. 7 contains the results of the GMDH fitting procedure to obtain the alternative models selecting of MCI versus (AD+control) patients:

### Example 6

### Visualization of One Possible Pair of Peptide Analytes for the Prediction of AD Cases

Out of the list of 4 parametric models it can be shown that the sub-model containing peptides JFJEPTRK and SJFTDJEAENDVJHCVAFAVPK already achieves quite good predictions for the AD versus MCI+control case. The sensitivity and specificity for this panel were 0.37 and 0.97 respectively.

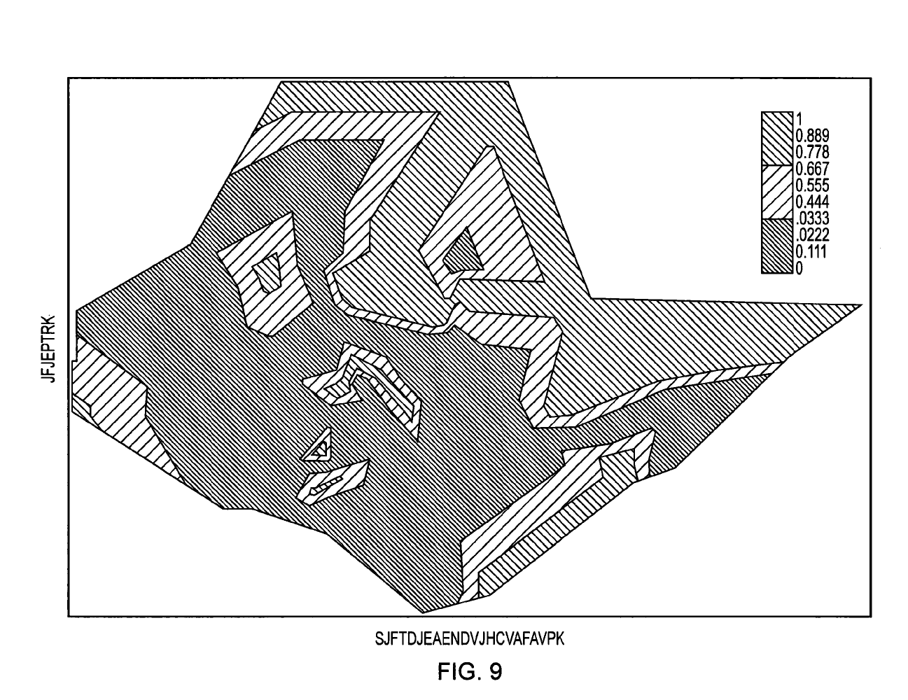

The diagram of FIG. 9 is a contour plot illustrating the density of AD patients using these two variables.

### REFERENCES

A. G. Ivakhnenko. Heuristic Self-Organization in Problems of Engineering Cybernetics. Automatica 6: pp. 207-219, 1970
A. G. Ivakhnenko. Polynomial Theory of Complex System. IEEE Trans. on Systems, Man and Cybernetics, Vol. SMC-1, No. 4, Oct. 1971, pp. 364-378.
S. J. Farlow. Self-Organizing Methods in Modelling: GMDH Type Algorithms. New-York, Bazel: Marcel Decker Inc., 1984, 350 p.
H. R. Madala, A. G. Ivakhnenko. Inductive Learning Algorithms for Complex Systems Modeling. CRC Press, Boca Raton, 1994.

1-27. (canceled)

28. A method of diagnosing Alzheimer's disease or mild cognitive impairment (MCI) in a subject, the method comprising detecting a panel of biomarkers in a tissue or body fluid sample from said subject, wherein said panel of biomarkers comprises two or more peptides selected from Table 2, Table 3 or Table 4.

29. The method according to claim 28, wherein:
(a) the presence of said two or more peptides in said sample is indicative of the patient having Alzheimer's disease or MCI;
(b) the amount or concentration of said two or more peptides in said sample, as compared to a reference value for said two or more peptides, is indicative of the subject having Alzheimer's disease or MCI; or
(c) a change in amount or concentration of said two or more peptides, as compared to a reference value for said two or more peptides, is indicative of the subject having Alzheimer's disease or MCI.

30. A method for diagnosing a form of dementia selected from the group consisting of Alzheimer's disease and mild cognitive impairment (MCI) in a subject, the method comprising:
(a) obtaining a tissue or body fluid sample from a patient,
(b) optionally treating the sample to enhance at least one marker protein selected from Table 1;
(c) treating the sample with the enzyme trypsin to create a plurality of peptides derived from said marker proteins;
(d) detecting a panel of biomarkers, said panel comprising two or more peptides selected from Table 2, Table 3 or Table 4;
(e) determining a value for the amount or concentration, presence, absence or change in said panel of biomarkers as compared to a reference value for said panel of biomarkers,
(f) diagnosing said subject based on the determined value.

31. A method according to claim 29, wherein said reference value is:
(i) derived from a previous sample taken from said subject; or
(ii) derived from a population of subjects.

32. The method according to claim 29, wherein said reference value is a pre-determined value in the form of an accessible database, preferably said database comprises Table 2, Table 3 or Table 4.

33. The method according to claim 29, wherein said reference value discriminates between:
(i) Alzheimer's disease and MCI or normal; or
(ii) MCI and Alzheimer's disease and normal.

34. The method according to claim 28, wherein the tissue or body fluid sample is a urine, blood, plasma, serum, saliva or cerebro-spinal fluid sample.

35. The method according to claim 28, wherein the biomarkers are detected in the sample using:
(i) specific antibodies, 2D gel electrophoresis or by mass spectrometry; or
(ii) antibodies or fragments thereof specific for two or more peptides in the panel of biomarkers.

36. The method according to claim 35, wherein the sample is pretreated with antibodies specific to at least one of the biomarker proteins listed in Table 1 in order to enrich the sample.

37. The method according to claim 28, wherein the two or more peptides of the biomarker panel are detected by mass spectrometry.

38. The method according to claim 37, wherein determining the amount or concentration of the two or more peptides is performed by Selected Reaction Monitoring (SRM) using one or more transitions for the peptides; and comparing the peptide levels in the sample being tested with peptide levels previously determined to represent Alzheimer's disease or MCI or non-demented patients.

39. A method according to claim 38, wherein comparing the peptide levels includes determining the amount or concentration of peptides in the sample with known amounts or concentrations of corresponding synthetic peptides, wherein the synthetic peptides are identical in sequence to the peptides obtained from the sample except for a label.

40. The method according to claim 39, wherein the label is a tag of a different mass or a heavy isotope.

41. The method according to claim 28, wherein the panel of biomarkers comprises:
(i) three or more peptides selected from Table 2, Table 3 or Table 4; or
(ii) a combination of peptides selected from the group of peptide combinations Y1 to Y30 as shown in FIG. 5; or
(iii) a combination of peptides selected from the group of peptide combinations Y1 to Y30 as shown in FIG. 7.

42. The method according to claim 41, wherein the panel of biomarkers comprises a combination of peptides selected from the group of peptide combinations Y1 to Y30 as shown in FIG. 7, wherein:
(i) Y1=VYAYYNJEESCTR*p1+TAGWNJPMGJJYNK*p2+SSSKDNJR*p3+DSSVPNTGTAR*p4; or
(ii) Y1=EFN_AETFTFHADICTISEK*p1+QGIPFFGQVR*p2−TEGDGVYTINDK*p3+NTCNHDEDTWVECEDPFDIR*p4+SSSKDNIR*p5−NIIDRQDPPSVVVTSHQAPGEK*p6.

43. The method according to claim 42, wherein a composite score “Y” is computed based on the relative abundance of each peptide in the panel of biomarkers relative to a reference control peptide; wherein an increased value of Y indicates a diagnosis of Alzheimer's disease or MCI, and optionally, the composite score “Y” is calculated according to the polynomial model
$$Y ( {x}_{1} , … , {x}_{n} ) = {a}_{0} + {\sum }_{i = 1}^{n} {a}_{i} {x}_{i} .$$

44. The method according to claim 43, wherein a total score value of >0.5 is indicative of the subject having Alzheimer's disease or MCI.

45. A kit for use in carrying out the method of claim 28, said kit comprising:
(a) two or more synthetic peptides corresponding to two or more peptides selected from Table 2, Table 3 or Table 4;
(b) two or more antibodies specific for the two or more peptides in the panel of biomarkers; or
(c) two or more binding members capable of specifically binding to said two or more peptides in the panel of biomarkers; said binding member optionally being fixed to a solid support.


SEQUENCE LISTING (92 sequences)

SEQ ID NO: 1 - protein, 8 aa, Homo sapiens
FYSEKECR

SEQ ID NO: 2 - protein, 9 aa, Homo sapiens
  VARIANT (3)..(3): Xaa is Ile or Leu
MFXSFPTTK

SEQ ID NO: 3 - protein, 10 aa, Homo sapiens
  VARIANT (1)..(1): Xaa is Ile or Leu
XGMFNXQHCK

SEQ ID NO: 4 - protein, 19 aa, Homo sapiens
EGKQVGSGVTTDQVQAEAK

SEQ ID NO: 5 - protein, 14 aa, Homo sapiens
  VARIANT (1)..(1): Xaa is Ile or Leu
XAYGTQGSSGYSXR

SEQ ID NO: 6 - protein, 12 aa, Homo sapiens
  VARIANT (10)..(10): Xaa is Ile or Leu
TQVNTQAEQXRR

SEQ ID NO: 7 - protein, 6 aa, Homo sapiens
  VARIANT (1)..(1): Xaa is Ile or Leu
XVSANR

SEQ ID NO: 8 - protein, 20 aa, Homo sapiens
  VARIANT (1)..(1): Xaa is Ile or Leu
XSXTGTYDXKSVXGQXGXTK

SEQ ID NO: 9 - protein, 9 aa, Homo sapiens
FMQAVTGWK

SEQ ID NO: 10 - protein, 11 aa, Homo sapiens
  VARIANT (3)..(3): Xaa is Ile or Leu
YGXVTYATYPK

SEQ ID NO: 11 - protein, 18 aa, Homo sapiens
  VARIANT (5)..(6): Xaa is Ile or Leu
VRVEXXHNPAFCSXATTK

SEQ ID NO: 12 - protein, 15 aa, Homo sapiens
  VARIANT (2)..(2): Xaa is Ile or Leu
HXEVDVWVXEPQGXR

SEQ ID NO: 13 - protein, 10 aa, Homo sapiens
  VARIANT (8)..(8): Xaa is Ile or Leu
SFFPENWXWR

SEQ ID NO: 14 - protein, 9 aa, Homo sapiens
REQPGVYTK

SEQ ID NO: 15 - protein, 9 aa, Homo sapiens
  VARIANT (2)..(2): Xaa is Ile or Leu
TXPEPCHSK

SEQ ID NO: 16 - protein, 11 aa, Homo sapiens
  VARIANT (1)..(1): Xaa is Ile or Leu
XGMFNXQHCKK

SEQ ID NO: 17 - protein, 10 aa, Homo sapiens
  VARIANT (2)..(2): Xaa is Ile or Leu
NXAVSQVVHK

SEQ ID NO: 18 - protein, 22 aa, Homo sapiens
  VARIANT (6)..(7): Xaa is Ile or Leu
QGPVNXXSDPEQGVEVTGQYER

SEQ ID NO: 19 - protein, 18 aa, Homo sapiens
  VARIANT (2)..(2): Xaa is Ile or Leu
SXGECCDVEDSTTCFNAK

SEQ ID NO: 20 - protein, 19 aa, Homo sapiens
  VARIANT (4)..(4): Xaa is Ile or Leu
QVQXVQSGGGXVKPGGSXR

SEQ ID NO: 21 - protein, 8 aa, Homo sapiens
DQGHGHQR

SEQ ID NO: 22 - protein, 12 aa, Homo sapiens
  VARIANT (8)..(9): Xaa is Ile or Leu
SHKWDREXXSER

SEQ ID NO: 23 - protein, 11 aa, Homo sapiens
  VARIANT (1)..(1): Xaa is Ile or Leu
XTXXSAXVETR

SEQ ID NO: 24 - protein, 9 aa, Homo sapiens
  VARIANT (5)..(6): Xaa is Ile or Leu
YYTYXXMNK

SEQ ID NO: 25 - protein, 11 aa, Homo sapiens
  VARIANT (3)..(3): Xaa is Ile or Leu
DQXTCNKFDXK

SEQ ID NO: 26 - protein, 10 aa, Homo sapiens
  VARIANT (3)..(3): Xaa is Ile or Leu
SVXGQXGXTK

SEQ ID NO: 27 - protein, 9 aa, Homo sapiens
  VARIANT (2)..(2): Xaa is Ile or Leu
SXTSCXDSK

SEQ ID NO: 28 - protein, 6 aa, Homo sapiens
EKGYPK

SEQ ID NO: 29 - protein, 11 aa, Homo sapiens
  VARIANT (10)..(10): Xaa is Ile or Leu
VRESDEETQXK

SEQ ID NO: 30 - protein, 16 aa, Homo sapiens
  VARIANT (2)..(3): Xaa is Ile or Leu
EXXSVDCSTNNPSQAK

SEQ ID NO: 31 - protein, 15 aa, Homo sapiens
  VARIANT (9)..(10): Xaa is Ile or Leu
HPYFYAPEXXFFAKR

SEQ ID NO: 32 - protein, 15 aa, Homo sapiens
  VARIANT (1)..(1): Xaa is Ile or Leu
XCMGSGXNXCEPNNK

SEQ ID NO: 33 - protein, 17 aa, Homo sapiens
  VARIANT (4)..(4): Xaa is Ile or Leu
VKDXATVYVDVXKDSGR

SEQ ID NO: 34 - protein, 8 aa, Homo sapiens
  VARIANT (7)..(7): Xaa is Ile or Leu
SSSKDNXR

SEQ ID NO: 35 - protein, 14 aa, Homo sapiens
  VARIANT (6)..(6): Xaa is Ile or Leu
TAGWNXPMGXXYNK

SEQ ID NO: 36 - protein, 6 aa, Homo sapiens
SEVAHR

SEQ ID NO: 37 - protein, 11 aa, Homo sapiens
DSSVPNTGTAR

SEQ ID NO: 38 - protein, 6 aa, Homo sapiens
EAVSGR

SEQ ID NO: 39 - protein, 13 aa, Homo sapiens
  VARIANT (7)..(7): Xaa is Ile or Leu
VYAYYNXEESCTR

SEQ ID NO: 40 - protein, 22 aa, Homo sapiens
  VARIANT (2)..(2): Xaa is Ile or Leu
SXFTDXEAENDVXHCVAFAVPK

SEQ ID NO: 41 - protein, 21 aa, Homo sapiens
  VARIANT (6)..(6): Xaa is Ile or Leu
AGAFCXSEDAGXGXSSTASXR

SEQ ID NO: 42 - protein, 8 aa, Homo sapiens
  VARIANT (1)..(1): Xaa is Ile or Leu
XFXEPTRK

SEQ ID NO: 43 - protein, 16 aa, Homo sapiens
  VARIANT (2)..(2): Xaa is Ile or Leu
SXDFTEXDVAAEKXDR

SEQ ID NO: 44 - protein, 11 aa, Homo sapiens
HVVPNEVVVQR

SEQ ID NO: 45 - protein, 13 aa, Homo sapiens
  VARIANT (4)..(4): Xaa is Ile or Leu
VEPXRAEXQEGAR

SEQ ID NO: 46 - protein, 15 aa, Homo sapiens
  VARIANT (10)..(11): Xaa is Ile or Leu
RHPYFYAPEXXFFAK

SEQ ID NO: 47 - protein, 6 aa, Homo sapiens
QHEKER

SEQ ID NO: 48 - protein, 12 aa, Homo sapiens
  VARIANT (9)..(9): Xaa is Ile or Leu
TEGDGVYTXNDK

SEQ ID NO: 49 - protein, 8 aa, Homo sapiens
  VARIANT (6)..(6): Xaa is Ile or Leu
DKCEPXEK

SEQ ID NO: 50 - protein, 9 aa, Homo sapiens
  VARIANT (5)..(6): Xaa is Ile or Leu
DNCCXXDER

SEQ ID NO: 51 - protein, 9 aa, Homo sapiens
  VARIANT (4)..(4): Xaa is Ile or Leu
DGYXFQXXR

SEQ ID NO: 52 - protein, 7 aa, Homo sapiens
GPTQEFK

SEQ ID NO: 53 - protein, 14 aa, Homo sapiens
  VARIANT (7)..(8): Xaa is Ile or Leu
KCSTSSXXEACTFR

SEQ ID NO: 54 - protein, 21 aa, Homo sapiens
  VARIANT (8)..(8): Xaa is Ile or Leu
MPCAEDYXSVVXNQXCVXHEK

SEQ ID NO: 55 - protein, 6 aa, Homo sapiens
TTVMVK

SEQ ID NO: 56 - protein, 17 aa, Homo sapiens
  VARIANT (8)..(8): Xaa is Ile or Leu
VFDEFKPXVEEPQNXXK

SEQ ID NO: 57 - protein, 21 aa, Homo sapiens
  VARIANT (13)..(13): Xaa is Ile or Leu
EFNAETFTFHADXCTXSEKER

SEQ ID NO: 58 - protein, 12 aa, Homo sapiens
  VARIANT (3)..(3): Xaa is Ile or Leu
SGXSTGWTQXSK

SEQ ID NO: 59 - protein, 20 aa, Homo sapiens
  VARIANT (19)..(19): Xaa is Ile or Leu
NTCNHDEDTWVECEDPFDXR

SEQ ID NO: 60 - protein, 11 aa, Homo sapiens
  VARIANT (5)..(5): Xaa is Ile or Leu
SASDXTWDNXK

SEQ ID NO: 61 - protein, 14 aa, Homo sapiens
  VARIANT (9)..(9): Xaa is Ile or Leu
VPQVSTPTXVEVSR

SEQ ID NO: 62 - protein, 8 aa, Homo sapiens
AEFAEVSK

SEQ ID NO: 63 - protein, 8 aa, Homo sapiens
  VARIANT (5)..(5): Xaa is Ile or Leu
RPSGXPER

SEQ ID NO: 64 - protein, 19 aa, Homo sapiens
  VARIANT (2)..(2): Xaa is Ile or Leu
EXKEQQDSPGNKDFXQSXK

SEQ ID NO: 65 - protein, 11 aa, Homo sapiens
  VARIANT (8)..(10): Xaa is Ile or Leu
HPDYSVVXXXR

SEQ ID NO: 66 - protein, 9 aa, Homo sapiens
TPVSDRVTK

SEQ ID NO: 67 - protein, 19 aa, Homo sapiens
  VARIANT (2)..(2): Xaa is Ile or Leu
NXREGTCPEAPTDECKPVK

SEQ ID NO: 68 - protein, 13 aa, Homo sapiens
  VARIANT (9)..(9): Xaa is Ile or Leu
TEGDGVYTXNDKK

SEQ ID NO: 69 - protein, 22 aa, Homo sapiens
  VARIANT (2)..(3): Xaa is Ile or Leu
NXXDRQDPPSVVVTSHQAPGEK

SEQ ID NO: 70 - protein, 13 aa, Homo sapiens
  VARIANT (4)..(4): Xaa is Ile or Leu
DVFXGMFXYEYAR

SEQ ID NO: 71 - protein, 19 aa, Homo sapiens
  VARIANT (13)..(13): Xaa is Ile or Leu
EFNAETFTFHADXCTXSEK

SEQ ID NO: 72 - protein, 10 aa, Homo sapiens
  VARIANT (1)..(1): Xaa is Ile or Leu
XDAQASFXPK

SEQ ID NO: 73 - protein, 7 aa, Homo sapiens
GNQESPK

SEQ ID NO: 74 - protein, 10 aa, Homo sapiens
  VARIANT (3)..(3): Xaa is Ile or Leu
QGXPFFGQVR

SEQ ID NO: 75 - protein, 15 aa, Homo sapiens
  VARIANT (1)..(1): Xaa is Ile or Leu
XRTEGDGVYTXNDKK

SEQ ID NO: 76 - protein, 8 aa, Homo sapiens
  VARIANT (1)..(1): Xaa is Ile or Leu
XSVXRPSK

SEQ ID NO: 77 - protein, 20 aa, Homo sapiens
  VARIANT (12)..(12): Xaa is Ile or Leu
QSNNKYAASSYXSXTPEQWK

SEQ ID NO: 78 - protein, 29 aa, Homo sapiens
  VARIANT (5)..(6): Xaa is Ile or Leu
DQFNXXVFSTEATQWRPSXVPASAENVNK

SEQ ID NO: 79 - protein, 6 aa, Homo sapiens
  VARIANT (3)..(4): Xaa is Ile or Leu
EVXXPK

SEQ ID NO: 80 - protein, 11 aa, Homo sapiens
VGFYESDVMGR

SEQ ID NO: 81 - protein, 12 aa, Homo sapiens
  VARIANT (9)..(11): Xaa is Ile or Leu
RHPDYSVVXXXR

SEQ ID NO: 82 - protein, 10 aa, Homo sapiens
  VARIANT (2)..(2): Xaa is Ile or Leu
VXVDHFGYTK

SEQ ID NO: 83 - protein, 9 aa, Homo sapiens
DYFMPCPGR

SEQ ID NO: 84 - protein, 8 aa, Homo sapiens
  VARIANT (1)..(2): Xaa is Ile or Leu
XXEXTGPK

SEQ ID NO: 85 - protein, 19 aa, Homo sapiens
EFNAETFTFHADICTISEK

SEQ ID NO: 86 - protein, 10 aa, Homo sapiens
QGIPFFGQVR

SEQ ID NO: 87 - protein, 12 aa, Homo sapiens
TEGDGVYTINDK

SEQ ID NO: 88 - protein, 20 aa, Homo sapiens
NTCNHDEDTWVECEDPFDIR

SEQ ID NO: 89 - protein, 8 aa, Homo sapiens
SSSKDNIR

SEQ ID NO: 90 - protein, 22 aa, Homo sapiens
NIIDRQDPPSVVVTSHQAPGEK

SEQ ID NO: 91 - protein, 13 aa, Homo sapiens
VYAYYNIEESCTR

SEQ ID NO: 92 - protein, 14 aa, Homo sapiens
TAGWNIPMGIIYNK